(19) United States
(12) Patent Application Publication
(10) Pub. No.: US 2004/0199937 A1
(43) Pub. Date: Oct. 7, 2004

(54) Plastid Transformation of Tobacco Suspension Cells

(75) Inventors: Camri Langbecker, Arnold, MO (US); Jeffrey M. Staub, Wildwood, MO (US); Guangning Ye, Chesterfield, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(73) Assignee: MONSANTO TECHNOLOGY LLC, St. Louis, MO (US)

(21) Appl. No.: 10/708,882

(22) Filed: Mar. 30, 2004

Related U.S. Application Data
(60) Provisional application No. 60/320,078, filed on Apr. 1, 2003.

(51) Int. Cl. C12N 15/82; C12N 15/87

(52) U.S. Cl. 800/278; 800/293

(57) ABSTRACT

A method is provided for transforming undeveloped plastids in tobacco suspension culture to produce a transplastomic plant.

This application claims priority to US Provisional Application 60/320,078, filed Apr. 1, 2003, herein incorporated by reference.

### BACKGROUND OF INVENTION

This invention relates to the application of genetic engineering techniques to plants. Specifically, the invention relates to compositions and methods for transformation of nucleic acid sequences into plant cell plastids.

Molecular biological techniques have enabled researchers to introduce pieces of DNA from one organism to another organism. Such techniques, referred to as recombinant DNA technology, have positively impacted the areas of medicine and agriculture. Conventional cloning methods have enabled the introduction of new pharmaceuticals and improved crops of agricultural importance. As the need for the introduction of multiple pieces of DNA and larger fragments of DNA into numerous target hosts increases, the need for novel cloning strategies increases accordingly.

The plastids of higher plants are an attractive target for genetic engineering. Plant plastids (chloroplasts, amyloplasts, etioplasts, chromoplasts, etc.) are the major biosynthetic centers that in addition to photosynthesis are responsible for production of industrially important compounds such as amino acids, complex carbohydrates, fatty acids, and pigments. Plastids are derived from a common precursor known as a proplastid, and thus the plastids present in a given plant species all have the same genetic content. Plant cells contain 500-10,000 copies of a small 120-160 kilobase circular genome, each molecule of which has a large (approximately 25 kb) inverted repeat. Thus, it is possible to engineer plant cells to contain up to 20,000 copies of a particular gene of interest, which potentially can result in very high levels of foreign gene expression.

Stable chloroplast transformation in higher plants has been demonstrated in tobacco (Svab et al., Proc. Natl. Acad. Sci. USA 87, 8526-8530,1990; Svab and Maliga, Proc. Natl. Acad. Sci. USA 90:913-917, 1993) and is used as a tool to answer fundamental questions in plastid biology or for over-expression of recombinant proteins of agronomic importance or potential therapeutic use. More recently, chloroplast transformation has been achieved in additional dicot plant species, including Arabidopsis (Sikdar et al., Plant Cell Reports. 18, 20-24, 1998), potato (Sidorov et al., The Plant Journal. 19, 209-216, 1999), tomato (Ruf et al., Nature Biotech. 19, 870-875, 2001), Lesquerella (Skarjinskaia et al., Transgenic Research PC1197, 1-8, 2002), petunia and canola. In each case, leaf chloroplasts have been used as the target for transformation because of abundance of the organelle in this tissue and the large plastid genome copy number in chloroplasts. A typical leaf cell contains as many as 100 chloroplasts per cell with up to 100 genome copies per chloroplast.

The present invention provides an efficient and reproducible procedure for stable plastid transformation of dark-grown tobacco suspension cells. This transformation system has a number of useful advantages, including easy maintenance of stock cultures and the ability for high throughput with less labor and more consistency than observed with leaf material.

### SUMMARY OF INVENTION

Novel methods for producing transplastomic plants from suspension cell cultures are provided. The invention described herein provides a method for obtaining stably transformed homoplasmic plants by growing suspension cell cultures and transforming the plastids with a heterologous nucleic acid sequence containing at least a selectable marker and, preferably a gene of interest conferring a desired phenotype to the plant. Plant cells containing transformed plastids are identified and selected and the transformed cells amplified. Transplastomic plants are regenerated therefrom. In a preferred embodiment, suspension cells that have been grown under dark lighting conditions from tobacco are used. Also provided herein are plants whose plastids are transformed to contain a heterologous nucleic acid sequence conferring a desired trait or outcome to the resulting transgenic plant.

### DETAILED DESCRIPTION

Definitions

In order to provide a clear and consistent understanding of the specification and the claims, including the scope given to such terms, the following definitions are provided.

A first nucleic acid sequence is “operably linked” with a second nucleic acid sequence when the sequences are arranged so that the first nucleic acid sequence affects the function of the second nucleic acid sequence. Preferably, the two sequences are part of a single contiguous nucleic acid molecule and more preferably are adjacent. For example, a promoter is operably linked to a gene if the promoter regulates or mediates transcription of the gene in a cell.

A “recombinant” nucleic acid is made by an artificial combination of two otherwise separated segments of sequence, e.g., by chemical synthesis or by the manipulation of isolated segments of nucleic acids by genetic engineering techniques. Techniques for nucleic-acid manipulation are well known (see for example, Sambrook et al., Molecular Cloning: A Laboratory Manual, Cold Spring Harbor Press, 1989; Maliga et al., Methods in Plant Molecular Biology, Cold Spring Harbor Press, 1995; Birren et al., Genome Analysis: volume 1, Analyzing DNA, (1997), volume 2, Detecting Genes, (1998), volume 3, Cloning Systems, (1999) volume 4, Mapping Genomes, (1999), Cold Spring Harbor, N.Y.).

A “synthetic nucleic acid sequence” can be designed and chemically synthesized for enhanced expression in particular host cells and for the purposes of cloning into appropriate vectors. Host cells often display a preferred pattern of codon usage (Campbell et al., Plant Physiol. 92:1-11, 1990). Synthetic DNAs designed to enhance expression in a particular host should therefore reflect the pattern of codon usage in the host cell. Computer programs are available for these purposes including but not limited to the “BestFit” or “Gap” programs of the Sequence Analysis Software Package, Genetics Computer Group, Inc., University of Wisconsin Biotechnology Center, Madison, Wis. 53711.

“Amplification” of nucleic acids or “nucleic acid reproduction” refers to the production of additional copies of a nucleic acid sequence and is carried out using polymerase chain reaction (PCR) technologies. A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. In PCR, a primer refers to a short oligonucleotide of defined sequence that is annealed to a DNA template to initiate the polymerase chain reaction.

“Transformed”, “transfected”, or “transgenic” refers to a cell, tissue, organ, or organism into which has been introduced a foreign nucleic acid, such as a recombinant vector. Preferably, the introduced nucleic acid is integrated into the genomic DNA of the recipient cell, tissue, organ or organism such that the introduced nucleic acid is inherited by subsequent progeny. A “transgenic” or “transformed” cell or organism also includes progeny of the cell or organism and progeny produced from a breeding program employing such a “transgenic” plant as a parent in a cross and exhibiting an altered phenotype resulting from the presence of a recombinant construct or vector.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression. Some genes can be transcribed into mRNA and translated into polypeptides (structural genes); other genes can be transcribed into RNA (e.g., rRNA, tRNA); and other types of genes function as regulators of expression (regulator genes).

“Expression” of a gene refers to the transcription of a gene to produce the corresponding mRNA and translation of this mRNA to produce the corresponding gene product, i.e., a peptide, polypeptide, or protein. Gene expression is controlled or modulated by regulatory elements including 5′ regulatory elements such as promoters.

“Genetic component” refers to any nucleic acid sequence or genetic element that may also be a component or part of an expression vector. Examples of genetic components include, but are not limited to, promoter regions, 5′ untranslated leaders, introns, genes, 3′ untranslated regions, and other regulatory sequences or sequences that affect transcription or translation of one or more nucleic acid sequences.

The terms “recombinant DNA construct,” “recombinant vector,” “expression vector” or “expression cassette” refer to any agent such as a plasmid, cosmid, virus, BAC (bacterial artificial chromosome), autonomously replicating sequence, phage, or linear or circular single-stranded or double-stranded DNA or RNA nucleotide sequence, derived from any source, capable of genomic integration or autonomous replication, comprising a DNA molecule in which one or more DNA sequences have been linked in a functionally operative manner using well-known recombinant DNA techniques.

As used herein, “heterologous” in reference to a nucleic acid is a nucleic acid that originates from a foreign species, or, if from the same species, is substantially modified from its native form in composition or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species, or, if from the same species, is substantially modified from its original form by deliberate human intervention.

As used herein, “recombinant” includes reference to a cell or vector that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

By “host cell” is meant a cell that contains a vector and supports the replication, transcription, or transcription and translation (expression) of the expression construct. Host cells for use in the present invention can be prokaryotic cells, such as E. coli, or eukaryotic cells such as yeast, plant, insect, amphibian, or mammalian cells. Preferably, host cells are plant cells.

As used herein, the term “plant” includes reference to whole plants, plant organs (for example, leaves, stems, roots, etc.), seeds, and plant cells and progeny of same. Plant cell, as used herein, includes, without limitation, seed suspension cultures, embryos, meristematic regions, callus tissue, leaves, roots, shoots, gametophytes, sporophytes, pollen, and microspores. The class of plants that can be used in the methods of the present invention is generally as broad as the class of higher plants amenable to transformation techniques.

As used herein, “transgenic plant” includes reference to a plant that comprises within its nuclear or plastid genome a heterologous polynucleotide. Generally, the heterologous polynucleotide is stably integrated within the nuclear genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “transplastomic” refers to a plant cell having a heterologous nucleic acid introduced into the plant cell plastid. The introduced nucleic acid may be integrated into the plastid genome or may be contained in an autonomously replicating plasmid. Preferably, the nucleic acid is integrated into the plant cell plastid's genome. A plant cell can be both transgenic and transplastomic.

The term “introduced” in the context of inserting a nucleic acid sequence into a cell, means “transfection” or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

The present invention describes a method for the production of transplastomic plant cells and transgenic plants derived therefrom using microprojectile bombardment of a suspension cell culture of the selected plant species. Successful plastid transformation includes several steps: a) engineering of plastid targeted constructs with a selectable marker suitable to select transplastomic cells; b) establishment of regenerable cell cultures containing plastids suitable for transformation; c) transformation of the regenerable cell cultures, usually by microprojectile bombardment; d) development of a selection scheme favorable for sorting out and selecting of transformed plastids from non-transformed plastids; and d) recovery of stable plastid transformants by regenerating the cell cultures into transplastomic plants.

In developing the constructs of the invention, the various fragments comprising the regulatory regions and open reading frame may be subjected to different processing conditions, such as ligation, restriction enzyme digestion, PCR, in vitro mutagenesis, linkers and adapters addition, and the like. Thus, nucleotide transitions, transversions, insertions, deletions, or the like, may be performed on the DNA that is employed in the regulatory regions or the nucleic acid sequences of interest for expression in the plastids. Methods for restriction digests, Klenow blunt end treatments, ligations, and the like are well known to those in the art and are described, for example, by Maniatis et al. (in Molecular cloning: a laboratory manual (1982) Cold Spring Harbor Laboratory Press, Cold Spring Harbor, N.Y.).

During the preparation of the constructs, the various fragments of DNA will often be cloned in an appropriate cloning vector, which allows for amplification of the DNA, modification of the DNA or manipulation by joining or removing of sequences, linkers, or the like. Normally, the vectors will be capable of replication in at least a relatively high copy number in E. coli. A number of vectors are readily available for cloning, including such vectors as pBR322, pUC series, M13 series, and pBluescript (Strategene; La Jolla, Calif.).

The constructs for use in the methods of the present invention are prepared to direct the expression of the nucleic acid sequences directly from the host plant cell plastid. Examples of such constructs and methods are known in the art and are generally described, for example, in Svab et al. (1990) Proc. Natl. Acad. Sci. USA 87:8526-8530 and Svab and Maliga (1993) Proc. Natl. Acad. Sci. USA 90:913-917 and in U.S. Pat. No. 5,693,507, herein incorporated by reference in its entirety.

The skilled artisan will recognize that any convenient element that is capable of initiating transcription in a plant cell plastid, also referred to as “plastid functional promoters,” can be employed in the constructs of the present invention. A number of plastid functional promoters are available in the art for use in the constructs and methods of the present invention. Such promoters include, but are not limited to, the promoter of the D1 thylakoid membrane protein, psbA (Staub et al. EMBO Journal, 12(2):601-606, 1993), and the 16S rRNA promoter region, Prrn (Svab and Maliga, Proc. Natl. Acad. Sci. USA 90:913-917, 1993). The expression cassette(s) can include additional elements for expression of the protein, such as transcriptional and translational enhancers, ribosome binding sites, and the like.

As translation is a limiting step for plastid transgene expression, a variety of translational control elements can be tested for efficacy. Efficient transgene translation will ensure that the markers used for selection of plastid transformed cells will function. Examples of such translational enhancing sequences include the heterologous bacteriophage gene 10 leader (G10L), the G10L including a downstream box sequence (DB), or a sequence encoding the first 14 amino acids from the green fluorescent protein (GFP) enhancer elements.

Regulatory transcript termination regions may be provided in the expression constructs of this invention as well. Transcript termination regions may be provided by any convenient transcription termination region derived from a gene source, for example, the transcript termination region that is naturally associated with the transcript initiation region. The skilled artisan will recognize that any convenient transcript termination region that is capable of terminating transcription in a plant cell may be employed in the constructs of the present invention.

The expression cassettes for use in the methods of the present invention also preferably contain additional nucleic acid sequences providing for the integration into the host plant cell plastid genome or for autonomous replication of the construct in the host plant cell plastid. Preferably, the plastid expression constructs contain regions of homology for integration into the host plant cell plastid. The regions of homology employed can target the constructs for integration into any region of the plastid genome; preferably the regions of homology employed target the construct to either the inverted repeat region of the plastid genome or the large single copy region. Where more than one construct is to be used in the methods, the constructs can employ the use of the regions of homology to target the insertion of the construct into the same or a different position of the plastid genome.

Additional expression cassettes can comprise any nucleic acid to be introduced into a host cell plastid by the methods encompassed by the present invention including, for example, DNA sequences or genes from another species, or even genes or sequences that originate with or are present in the same species, but are incorporated into recipient cells by genetic engineering methods rather than classical reproduction or breeding techniques. An introduced piece of DNA can be referred to as exogenous DNA. Exogenous as used herein is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

The plastids in dark-grown suspension cells are relatively small, undeveloped and dispersed throughout the cell. Dark-grown means that the cells are grown in complete darkness but all subculture manipulations are done in white light. Remarkably, confocal scanning microscopy indicates an approximately 50-fold difference in plastid volume between leaf cell chloroplasts and the undeveloped plastids in cell suspensions. Despite these obvious differences, in the present invention plastid transformation of the suspension cells is at least as efficient as the chloroplast-containing leaf system, with an average of 1-2 plastid transformants per bombarded shot. Furthermore, the number of transformants on each plate was consistent from experiment to experiment when optimal parameters were used, in contrast to the leaf transformation system, which is highly variable.

Suspension cell culture of plant cells is well-known in the art (i.e., for tobacco see Paszty and Lurquin, Arch Biochem Biophys 247: 211-220, 1986). One of skill in the art can readily devise a suspension cell culture appropriate for the desired plant system.

Introduction of foreign DNA into chloroplasts is achieved via the particle bombardment process, and integration of transgenes is mediated by homologous recombination through flanking plastid DNA in the transformation vector. Upon integration of the transgenes, stable transformation is achieved as a result of amplification and sorting of transgenic plastid genome copies with concomitant elimination of wild-type genomes under continued selective pressure. Elimination of wild-type genomes to yield homoplasmic lines is required for stability of the transgenes. In the tobacco leaf chloroplast transformation system, homoplasmic R0 plants or seedlings derived from these are typically the useful endpoint.

As exemplified herein, high-frequency plastid transformation in tobacco suspension cells was obtained by optimization of several factors, including growth rate, selection parameters, particle size and DNA concentration. As is well understood, a wide range of conditions would be appropriate even under less than optimal conditions. Cultures were used for plastid transformation experiments by subculturing of 10 mL of suspended cells into 40 mL of fresh medium weekly (1×/wk cells). The suspensions were also transferred at a ratio of 30 mL of suspended cells into 40 mL of fresh medium two times per week (2×/wk cells). A large increase in plastid transformation frequency was observed using the 2×/wk cultures cells that are bombarded only one day post transfer, as compared to the 1×/wk cultured cells bombarded at 3 days post subculture. A similar large increase in transformation frequency was observed when selected cultures were allowed to remain on antibiotic-containing medium rather than being frequently transferred to fresh selection medium.

The suspension cell system was also proven efficient for nuclear transformation, allowing direct comparison of bombardment parameters on the transformation efficiency in each case. The standard protocol for tobacco leaf chloroplast transformation utilizes 0.6 or 1 micron tungsten or gold particles for transformation, using a rupture-disc pressure of 1100 psi. Because plastids in cell suspensions have a significantly smaller volume than chloroplasts of leaf cells and are randomly distributed throughout the cell, we wanted to determine if particle size or bombardment pressure would influence efficiency of plastid transformation. Gold particles were chosen for this study because the commercially available bead preparations are very uniform in size as compared to tungsten preparations (M5 or M10 tungsten is the usual preparation). Particle sizes of 0.4, 0.6 and 1 micron, at two different rupture pressures, were tested. Also interesting, DNA concentration was not critical across a 200-fold range for either nuclear or plastid transformation, although a minimal threshold of DNA was required for efficient transformation in both cases.

Once obtained, plastid transformants were readily converted to homoplasmy in either cell colonies or in plants regenerated from those. This result indicates that there is no fundamental barrier to transformation of plastid types other than chloroplasts. With the additional consistency, ease of use and maintenance, and potential to circumvent the need for regenerated plants, the tobacco suspension cell system may also replace tobacco leaf in some cases as the vehicle for study of plastid gene function through transformation.

Because plant cells contain a large copy number of plastid genomes, an effective selectable marker and selection regime are very important for selecting homoplasmic transformants. The most widely used selectable marker is a chimeric bacterial-derived antibiotic resistance marker, aada, that confers resistance to spectinomycin and streptomycin (Svab and Maliga, Proc. Natl. Acad. Sci. USA 90:913-917, 1993, herein incorporated by reference in its entirety). Selection of transformants is based on greening and enhanced growth of resistant cells whereas sensitive cells are bleached and growth inhibited (Maliga, TIBTECH 11, 101-106, 1993). The CP4 gene that confers resistance to the herbicide glyphosate has also been used for selection in plastids. In the case of streptomycin, the antibiotic is not lethal to plant cells. Selection relies on green phenotype of resistant cells. In contrast, glyphosate is a plastid-lethal marker and destroys plastid membrane structure at the lethal concentration. Effective selection therefore may be facilitated by a sub-lethal selection scheme, as outlined in US Patent Application US20020042934, incorporated by reference herein.

### EXAMPLES

The following examples further illustrate the present invention. They are in no way to be construed as a limitation in scope and meaning of the claims.

### Example 1

### Initiation and Maintenance of Tobacco Suspension Cultures

Young, mature leaves from Nicotiana tabacum cv. Petit Havana plants grown in sterile tissue culture were used as starting material. Plants were raised from seed on Germination Medium [MS salts (Murashige and Skoog, Physiol. Plant 15, 473-497, 1962), B-5 vitamins (Gamborg et al. Exp. Cell. Res. 50, 151-158, 1968), 3% w/v sucrose, and 8 g/L TC agar] at 24° C. with a 16-hour light:8-hour dark photoperiod. Light intensity of 35-58 μE m⁻² sec⁻¹ was delivered by cool white fluorescent bulbs. Mature plants were propagated by placing cuttings onto fresh germination medium. Mature leaves were removed from plants about 4-6 weeks after propagation, cut into 0.5-1.0 cm squares and placed onto Callus Medium (MS salts, B-5 vitamins, 4 mg/L p-chlorophenoxyacetic acid, 5 μg/L kinetin, 3% sucrose, and 2.5 g/L Schweizer Hall gelling agent) and cultured at 25° C. in continuous dark for callus initiation. Translucent friable callus was separated from the leaf and transferred to fresh Callus Medium and sub-cultured every 3-4 weeks. Callus that had been propagated for 2-5 months was used to initiate liquid suspension cultures.

Suspension cell cultures were initiated by placing 20 grams of callus into 40 mL of filter sterilized Suspension Medium [MS salts (Murashige and Skoog, Physiol. Plant 15, 473-497, 1962), MS vitamins, 4 mg/L p-chlorophenoxyacetic acid, 5 pg/L kinetin, 0.2 g/L myolnositol, 0.15 g/L I-asparagine, 3% w/v sucrose]. Suspensions were cultured in the dark at 25° C. with shaking at 140-160 rpm. Subcultures of 1:1 with fresh medium were performed at weekly intervals for a period of 5-8 weeks with large clumps removed each week until a consistent suspension was obtained. Cultures were used for plastid transformation experiments by subculturing of 10 mL of suspended cells into 40 mL of fresh medium weekly (1×/wk cells). The suspensions were also transferred at a ratio of 30 mL of suspended cells into 40 mL of fresh medium two times per week (2×/wk cells). Culture density became consistent after approximately 5 weeks of subcultures at which time transformation experiments could be initiated.

Plastid Morphology in Tobacco Cell Suspensions

The dark-grown cells do not accumulate chlorophyll but do have a yellow appearance presumably due to accumulation of carotenoids or other pigments. Under light microscopy, the suspension cells appear to be predominantly small clumps of cells, with the nucleus apparent and large cytoplasm with no obvious vacuole. No other subcellular organelles are visible at this magnification. To determine the morphology of plastids in these cells, transmission electron microscopy was performed and compared to chloroplasts found in leaf mesophyll cells of in-vitro grown plants. The leaf cells contain chloroplasts with well developed membrane structure, are relatively uniform in size, and are localized around the periphery of the cell. In contrast, plastids in the tobacco suspension cells are randomly distributed throughout the cell cytoplasm, heterogeneous in size but smaller than in leaf cells, and have little to no apparent internal membrane structure. The plastids in suspension cells also contain large amounts of starch relative to leaf cell chloroplasts.

Confocal scanning microscopy was used to obtain a more precise measure of the diameter and volume of plastids in the different cell types. For this analysis, leaf tissue and suspension cells were derived from homoplasmic plastid transformed lines that express GFP and measurements were based on GFP fluorescence visualized by the confocal microscope. From this analysis, suspension cells had an average diameter and volume of 2.1 and 4.9 microns, respectively. In contrast, leaf cell chloroplasts had an average diameter and volume of 7.7 and 239 microns, respectively. Therefore, the average leaf chloroplast volume is approximately 50 fold greater than that of undeveloped plastids of suspension cells.

### Example 2

### Construction of Transformation Vectors

pMON30125 has been described previously (Sidorov et al., The Plant Journal. 19, 209-216, 1999) and was used for plastid transformation. This vector carries the aada selectable marker driven by psbA gene expression signals and the gfp gene driven by the Prm promoter and Trps16 expression cassette. For nuclear transformation, pMON38754 (Sidorov et al., The Plant Journal. 19, 209-216, 1999) was used. This vector carries the aada gene driven by the Figwort Mosaic Virus promoter, and the uida gene driven by the Cauliflower Mosaic Virus 35S promoter.

### Example 3

### Tobacco Suspension Transformation

The 2×/wk cells were used for bombardment experiments twenty-four hours post-subculture whereas the 1×/wk cells were used 3 days post-subculture. The suspension culture was then diluted to 0.5 mL cells per 10 mL of medium, using Suspension Medium, and dispersed into 0.25 mL aliquots.

Aliquots of suspended cells were transferred onto a 70 mm Whatman #1 filter using a Corning 500 mL 0.45 micron vacuum filter system. Small holes were introduced into the 0.45 micron filter to allow for more rapid adherence of the cells to the Whatman filter. The filter paper including the cells was then placed onto Bombardment Medium (MS salts, B-5 vitamins, 18.2 g/L mannitol, 18.2 g/L sorbitol, 0.1 mg/L 1-naphthaleneacetic acid, 1 mg/L 6-benzylaminopurine, 3% w/v sucrose, and 8 g/L TC agar) for 4 hours prior to bombardment. Bombardment was performed using the PDS1000-He biolistic particle delivery system (Bio-Rad, Laboratories, Hercules, Calif.) under a vacuum of 28 inches of mercury with the target shelf located 9 cm from the stopping screen. Particle size and rupture pressures were as outlined below. Approximately 20 hours after bombardment filters were transferred to Selection Medium (MS salts, B-5 vitamins, 3% w/v sucrose, 0.1 mg/L NAA, 1 mg/L BAP, 750 mg/L spectinomycin and 8 g/L TC agar) and cultured at 25° C. with a 16:8 hour photoperiod using cool white fluorescent lamps.

Particle size influences the frequency of plastid and nuclear transformation

The standard protocol for tobacco leaf chloroplast transformation utilizes M5 or M10 tungsten or 0.6 or 1 micron gold particles for transformation, using a rupture-disc pressure of 1100 psi. Because plastids in cell suspensions have a significantly smaller volume than chloroplasts of leaf cells and are randomly distributed throughout the cell, we wanted to determine if particle size or bombardment pressure would influence efficiency of plastid transformation. Gold particles were chosen for this study because the commercially available bead preparations are very uniform in size as compared to tungsten preparations. Particle sizes of 0.4, 0.6 and 1 micron, at two different rupture pressures, were tested.

No significant difference was observed when 0.6 and 1 micron particles were used for transformation, regardless of bombardment pressure used. In contrast, use of 0.4 micron particles resulted in a significantly higher (˜4-fold) transformation frequency. Interestingly, at this size of bead particle, the lower rupture-disc pressure used was more effective (1100 psi vs 1350 psi). In contrast, nuclear transformation was more efficient with the larger sized particles, indicating that the transformation processes for these two organelles must be optimized separately.

The Effect of DNA Concentration on Plastid and Nuclear Transformation

The amount of DNA used for bombardment of the tobacco cell suspensions was also investigated over a 200-fold range of DNA concentration, from 7.5 ng to 1.5 μg per bombardment. No differences were observed in transformation frequency in either plastid or nuclear transformation when the DNA concentration ranged from 75 ng to 1.5 μg per bombardment. However, in both transformation systems, the transformation frequency decreased significantly when only 7.5 ng DNA per bombardment was used. Plastid transformation frequency decreased approximately 10-fold whereas nuclear transformation frequency decreased as much as 30-fold.

### Example 4

### Selecton of Tobacco Suspension Cells and Regeneraton of Plants

After 3 or 6 weeks on Selection Medium, the filters were transferred to fresh medium. By 6 wk post-bombardment, putative plastid transformants could be identified by GFP fluorescence, and by 9 weeks putative plastid transformants could be identified by the green color of growing calli. Resistant calli were isolated at this time and transferred onto fresh Selection Medium without filter papers. Once the colonies had grown to 2-3 cm in diameter they were sampled for Southern blot analysis or cut into 3-4 mm slices and transferred flat-side down onto Selection Medium to stimulate shoot formation. Small shoots typically formed about 2 weeks after cutting. Shoots were removed and placed onto Rooting Medium (same as Selection Medium without BAP and NAA) for rooting.

Selection Conditions for Plastid Transformation

The standard protocol for selection of chloroplast transformants from tobacco leaf employs the antibiotic spectinomycin at selective levels of 500 mg/L. Transformants are selected in the light and typically arise as green shoots on plant regeneration medium. To determine the response of suspension cells to antibiotic treatment and identify selective levels for use in transformation experiments, the dark-grown cell suspensions were plated on spectinomycin levels up to 1500 mg/L in both the light and the dark.

In the light without antibiotic, the tobacco suspension cells turn green within ˜3 weeks and grow vigorously as large mounds of cells. In the presence of spectinomycin, both greening and growth is delayed. In contrast to leaf cells that bleach completely at 500 mg/L spectinomycin, cell suspensions continue to grow and numerous green mounds of cells arise. At 750 mg/L spectinomycin, cell growth was not dramatically inhibited but few green colonies formed, indicating this concentration may be optimal for selection of plastid transformants. Growth of dark-grown cells was only moderately inhibited at even the highest spectinomycin concentration tested, indicating that selection in the dark would be very difficult using this antibiotic.

Demonstration and Optimization of Stable Plastid Transformation

Preliminary plastid transformation experiments were designed to identify optimal cell-growth and antibiotic selection conditions for the cell suspension cultures. To determine the optimal growth rate of the cell suspensions, these were maintained under two different subculture regimes; transfers once weekly at a suspended cell:medium ratio (vol.:vol.) of 1:4 (1×/wk cells) and transfers twice weekly at 3:4 suspended cell:medium ratio (2×/wk cells). The 1×/wk cells were bombarded 3-days post-subculture, whereas the 2×/wk cells were bombarded 1-day post-subculture.

For selection of plastid transformants, cells were transferred one day after bombardment onto Selection Medium containing spectinomycin (750 mg/L) and incubated in the light. To test the effect of renewed selection medium on transformation frequency, half of the selected cells were transferred to fresh spectinomycin-medium after 3 weeks. At six weeks post-bombardment, all of the cells were transferred to fresh selection medium.

To select plastid transformed tobacco suspension cells, the pMON30125 plastid transformation vector used previously for selection of tobacco and potato plastid transformants was employed. This vector contains a chimeric aada gene that provides spectinomycin resistance and a GFP reporter gene used for early identification of plastid transformants (Sidorov et al., The Plant Journal. 19, 209-216, 1999). Transformation of leaf cells with this vector results in ˜1 transformant per bombardment in tobacco and ˜1 transformant per 5 bombardments in potato (Sidorov et al., The Plant Journal. 19, 209-216, 1999).

GFP fluorescence was used to identify putative plastid transformants on selection plates as early as six weeks post-bombardment. GFP fluorescence could be used to identify small colonies of transformed cells directly on plates within the background of nontransformed cells. The putative plastid transformed cells were then isolated onto fresh medium for further amplification prior to molecular analysis. Southern blot analysis was performed on GFP-positive samples to verify plastid transformation. This analysis verified that all tested GFP positive lines were due to insertion of the aadA and GFP transgenes into the plastid genome. As all GFP-positive lines were confirmed to be plastid transformants by molecular analysis, GFP fluorescence was used as an indicator of plastid transformation in subsequent experiments.

In a preliminary experiment to optimize growth and spectinomycin selection, a dramatic effect of subculture frequency and selection conditions on the frequency of plastid transformation was observed. Nearly all of the transgenic events (7 of 9) were recovered from 2×/wk subcultured cells that were allowed to remain on selection plates for six weeks prior to transfer to fresh selection medium. These conditions were therefore used in all subsequent experiments. On average, these optimal parameters routinely resulted in plastid transformation at ˜1-2 transformants per bombarded plate, a frequency similar to that reported for tobacco leaf transformation (Svab et al., Proc Natl Acad Sci U.S.A. 90, 913-7, 1993). However, it should be noted that transformation of cell suspensions also routinely resulted in transformants on nearly every bombarded plate, whereas tobacco leaf transformation is inconsistent and highly variable between experiments.

For comparison to plastid transformation frequencies, nuclear transformation of the 2×/wk cells was also performed. The pMON38754 nuclear transformation vector (Sidorov et al., The Plant Journal. 19, 209-216, 1999) that carries aada and uida genes under control of nuclear transcriptional control sequences, was used for transformation. Based on greening colonies and GUS staining in 9-12-week-old cell colonies selected using the same parameters as above, nuclear transformation frequency was ˜25-45 transformants per bombarded plate, or about 20 times more frequent than plastid transformation.

Homoplasmy can be Achieved in Cell Colonies or Regenerated Plants

For full utility of the tobacco suspension cell system, obtaining homoplasmic clones was important. Therefore, several clones identified initially by GFP fluorescence and subsequently by Southern blot analysis were further subcultured on two rounds of Selection Medium to attempt to obtain homoplasmic lines. These lines grew as cell colonies on solid medium, and shoots were subsequently regenerated from these colonies on the same media that were then transferred to Rooting Medium containing spectinomycin to form rooted plants. Several independent cell colonies and shoots were analyzed by Southern blot to determine if homoplasmy was achieved. Several lines carry exclusively the transgene insert with no apparent wild-type plastid genomes remaining. These results indicate that there is no barrier to achieving homoplasmy in plastid transformants derived from cell suspensions.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the invention has been described in detail for the purpose of illustration, it is understood that such detail is solely for that purpose, and variations can be made therein by those skilled in the art without departing from the spirit and scope of the invention which is defined by the following claims.

1. A method of producing transplastomic plants comprising the steps of:
(a) growing a suspension culture of plant cells containing plastids;
(b) transforming the plastids with a heterologous nucleic acid comprising at least a selectable marker;
(c) selecting for plant cells containing transformed plastids; and
(d) regenerating transplastomic plants from the plant cells containing transformed plastids.

2. The method of claim 1 in which the plastids are transformed by microparticle bombardment.

3. The method of claim 1 in which the suspension culture is grown in the dark.

4. The method of claim 1 in which the plant is tobacco.

5. The method of claim 1 in which the heterologous nucleic acid also comprises a gene of interest.
